(19) United States
(12) Patent Application Publication
(10) Pub. No.: US 2005/0221392 A1
(43) Pub. Date: Oct. 6, 2005

(54) Detection and isolation of cell populations from muscle using antibodies to fa1/dlk1

(76) Inventors: Charlotte Harken Jensen, Svendborg (DK); Borge Teisner, Vestergade (DK); Henrik Daa Schroder, Korup (DK)

Correspondence Address: BROWDY AND NEIMARK, P.L.L.C.;624 NINTH STREET, NW, SUITE 300, WASHINGTON, DC, 20001-5303, US

(21) Appl. No.: 10/508,429

(22) PCT Filed: Mar. 21, 2003
(86) PCT No.: PCT/DK03/00192

§ 371 Date: Apr. 27, 2005

Related U.S. Application Data
(60) Provisional application No. 60/366,421, filed on Mar. 21, 2002.

(30) Foreign Application Priority Data
Mar. 27, 2002 (DK) 2002-00481

(51) Int. Cl. G01N 33/53; G01N 33/567; C12N 5/08

(52) U.S. Cl. 435/7.2; 435/366

(57) ABSTRACT

The present invention relates to the use of antibodies recognizing Fetal Antigen-1 (FA1/dlk1) for the detection and isolation of cell populations in mammalian muscle. In one embodiment, myogenic progenitor cells are detected in developing, diseased or regenerating muscle. In another embodiment, muscle stem and progenitor myogenic progenitor cells are isolated from muscle tissue or from cultures containing muscle cells. The isolated cells may be used for transplantation, drug screening, production of cell type specific antibodies, and gene therapy and discovery. Transplantation of these cells may provide treatments for degenerative diseases of muscle, and for regeneration of muscle following trauma or ischemia such as myocardial infarction.

The present invention is a non-provisional of U.S.-provisional patent application Ser. No. 60/366,421 filed on 21 Mar. 2002 and claims priority from Danish patent application no. PA 2002 00481 filed on 27 Mar. 2002. All references cited in these applications or in the present application are hereby incorporated by reference in their entirety.

### FIELD OF INVENTION

The present invention concerns the use of FA1 antibodies for recognizing and isolating FA-1 expressing cells derived from mammalian muscle, which includes myogenic stem/progenitor cells.

### BACKGROUND OF THE INVENTION

Skeletal muscles of adult mammalian species exhibit a capacity to adapt to physiological demands such as growth, training, and injury. The processes by which these adaptations occur are attributed to a small population of mononuclear cells that is resident in adult skeletal muscle and has been referred to as satellite cells. Myogenic satellite cells have been the subject of a recent extensive review (Hawke & Garry 2001. J Appl Physiol 91: 534-551) Skeletal muscle fibers are terminally differentiated and the nuclei in these multinucleated cells are incapable of DNA synthesis or mitotic division. Increases in muscle fiber numbers or in numbers of muscle fiber nuclei are due to proliferation and subsequent differentiation of muscle precursor cells known as “myoblasts.” In adults, myoblasts remain as mitotically quiescent reserve precursor populations, which can, upon muscle injury, re-enter the cell cycle, undergo several rounds of proliferation, and subsequently differentiate and permanently exit from the cell cycle. Upon differentiation, differentiated myoblasts acquire the ability to fuse with one another or with preexisting muscle fibers, and also commence expression of a set of muscle-specific myofibrillary and contractile proteins. Quiescent myogenic progenitor cells are physically distinct from the adult myofibers as they reside in indentations between the sarcolemma and the basal lamina. In the case of muscle injury, some of these cells will remain as progenitor cells whereas others will differentiate into new muscle fibers. In response to stimuli such as myotrauma, myogenic progenitor cells become activated, proliferate, and express myogenic markers. Ultimately, these cells fuse to existing muscle fibers or fuse together to form new myofibers during regeneration of damaged skeletal muscle. Gussoni, (Nature 1999 Sep. 23;401(6751):390-39) found that intravenous injection of a population of muscle-derived stem cells into a mouse model of Duchenne's muscular dystrophy results in the incorporation of donor-derived nuclei into muscle, and the partial restoration of dystrophin expression in the affected muscle. The population containing the muscle stem cells was isolated by its low affinity for a particular dye. This intravenous route of administration may represent a means of treating degenerative diseases of muscle with a suitable population of myogenic progenitor cells.

Cardiac muscle, unlike skeletal muscle does not have the capacity to regenerate in response to injury. Populations of myogenic progenitor cells could therefore be used to regenerate cardiac muscle following myocardial infarction or ischemia (El Oakley R M Ann Thorac Surg 2001 May; 71(5): 1724-1733). The plasticity of the myogenic progenitor cell population suggests that these cells not only have a capacity for muscle regeneration but may also contribute to non-muscle lineages (Springer M L, et al. J Clin Invest. 2001 June; 107(11): 1355-6).

Some potential markers have been associated with myogenic progenitor cells. Satellite cells have been shown to express neural cell adhesion molecule (N-CAM) (Fidzianska A Folia Neuropathol 1995; 33(3): 125-128). Magnetic affinity cell sorting (MACS) has been used in the separation of NCAM-positive, cultured myogenic cells from normal and dystrophic dogs (Prattis S M Exp Cell Res 1993 October; 208(2): 453-64). Vascular adhesion molecule-1 (VCAM-1) is expressed on satellite cells and regenerating muscle fibres. (Jesse T L. J Cell Biol 140: 1265-1276, 1998) and M-cadherin, calcium-dependent cell adhesion molecule, has been suggested as a marker of the satellite cell population (Irintchev. Dev Dyn 199: 326-337, 1994). Similarly, Bcl-2 has been shown to be expressed in muscle stem cells (U.S. Pat. No. 6,337,184). Pax7 is a further maker of myogenic progenitor cells whose location is restricted to a sub-population of satellite cells (Seale P et al. Cell 2000;102:777-786).

The present invention provides a means of identifying and enriching for subpopulations of mononuclear myogenic progenitor cells that express FA1/dlk1 with or without selection for other markers.

Fetal antigen 1 (FA1) is one of the increasing numbers of proteins belonging to the epidermal growth factor (EGF)-super family that have been identified within the last decade. The protein contains 6 EGF-like repeats and displays a very similar primary structure and level of glycosylation in man, mouse and rat (Jensen C H, et al. Hum Reprod 1993 8(4), 635-641; Jensen C H, et al. Eur J Biochem 1994 225(1), 83-92. Bachmann E, et al. J Reprod Fertil 1996 107(2), 279-285. Krogh T N, et al. Eur J Biochem 1997 244(2), 334-342. Carlsson H E, et al. Biol Reprod 2000 63(1), 30-33.)

FA1 is synthesized as a larger transmembrane precursor and released from cells after proteolytic action of an unidentified enzyme. Several groups have described cDNA clones for this precursor, each assigning a new name for the cDNA depending on the species and tissue/cell type from which they isolated it. As a result, the FA1 precursor has been referred to as adrenal specific mRNA (human pG2 Helman L J. Nucleic Acids Res 1990 18(3), 685), delta-like (mouse and human dlk1 Laborda J, et al. J Biol Chem 1993 268(6), 3817-3820.), preadipocyte factor-1 (mouse, rat and bovine pref-1 Laborda J, et al. J Biol Chem 1993 268(6), 3817-3820. Smas C M, Cell 1993 73(4), 725-734; Carlsson C, et al. Endocrinology 1997 138(9), 3940; Fahrenkrug S C, Biochem Biophys Res Commun 1999 264(3), 662-667) and zona glomerulosa-specific factor (rat ZOG Okamoto, et al. Steroids 1997 62(1), 73-76). The official name for the gene encoding this membrane-associated protein is now delta-homologue 1, dlk11 (Gubina, et al. Cytogenet Cell Genet 1999 84(3-4), 206-207.), referring to the close resemblance between its EGF-repeats and those of the transmembrane protein Delta, which was originally described in Drosophila Melanogaster. Delta is one of the ligands for the Notch receptor and interactions between these membrane proteins are crucial for the development of various tissues [Artavanis-Tsakonas, Science 1995 268(5208), 225-232.]. The primary structure of dlk1 does not allow conclusions as to whether it is a ligand or receptor, but both the membrane-associated and the soluble form (i.e. FA1) of the DLK1 gene have been shown to be involved in the differentiation/proliferation processes of various cell types and act through autocrine/paracrine and juxtacrine intercellular signaling (reviewed by Laborda in Laborda J. Histol Histopathol 2000 15(1), 119-129.), the membrane-associated form possibly as a homodimer (Kaneta, J Immunol 2000 164(1), 256-264). Apart from being present in preadipocytes and stromal cells, the expression of FA1/dlk1 in adults seems to be associated with endocrine structures. FA1 has been localized in β-cells of the pancreatic islets of Langerhans (Jensen, Hum Reprod 1993 8(4), 635-641); Jensen, Eur J Biochem 1994 225(1), 83-92; Tornehave, Histochem Cell Biol 1996 106(6), 535], the adrenal gland (medulla and cortex) [Jensen, Hum Reprod 1993 8(4), 635-641], the somatotroph cells of the adenopituitary gland [Larsen, Lancet 1996 347(8995), 191], the sex hormone-producing Leydig cells of the testis, and theca interna and Hilus cells of the ovary [Jensen, Mol Hum Reprod 1999 5(10), 908]. FA1 has also been demonstrated in tumors [Jensen, Eur J Biochem 1994 225(1), 83-92; Tornehave, Histochem Cell Biol 1996 106(6), 535; Harken Jensen, Tumour Biol 1999 20(5), Jensen, Mol Hum Reprod 1999 5(10), 908-913] including Small Cell Lung Cancer, pheochromocytomas and neuroblastomas.

Although FA1 expression has been observed in fetal muscle (Floridon et al., Differentiation 2000 66(1), 49-59) it was only noted to be expressed in multi-nucleated myotubes of fetal skeletal muscles. Fully differentiated muscle cells were FA1 negative. Cardiac and smooth muscles were also FA1 negative. There was no evidence of the antigen in mononuclear myogenic progenitor cells. Furthermore, to our knowledge there are no published reports of FA1 expression in muscle stem- and progenitor cultures derived from mammalian muscle.

Antibodies to FA1 have been used previously for cell sorting. Bauer, (Molecular And Cellular Biology, p. 5247-5255 Vol. 18, No. 9) used anti-dlk1 polyclonal antiserum for dlk1 detection and flow cytometry analysis of detached stromal cells and pre-B cells. Garćes (Differentiation 1999 64:103-114) used Pref-1 antibodies for flow cytometry analysis and cell sorting of preadipocytes and their differentiated progeny.

### BRIEF DESCRIPTION OF THE INVENTION

The present invention concerns the use of antibodies that recognize the FA1 antigen that is expressed as a membrane-associated protein in specific populations of cells of mammalian muscle and in cultures containing mammalian muscle stem- and progenitor cells. An example of an antibody used in the invention is the mouse monoclonal FA1 antibody (clone 142.2) or a mono-specific polyclonal anti-FA1 antibody. The FA1 antibody binds to a population of mononuclear cells and immature muscle cells present in mammalian muscle and to sub-populations of cells in cultures containing and/or derived from muscle stem- and progenitor cells. The term mammalian includes any mammalian species, including mouse, rat, domestic animals, and preferably human beings.

Accordingly, there is provided a method for obtaining a cell population enriched in cells selected from the group consisting of muscle mononuclear cells, satellite cells, muscle stem cells, and muscle progenitor cells, said method comprising the steps of
a) providing a population of cells selected from the group consisting of: a mixed population of mammalian muscle cells, and cultures containing or derived from muscle stem- and/or progenitor cells,
b) contacting said population with labeled antibodies which bind specifically to FA1/dlk1,
c) selecting cells labelled with the FA1 antibody.

One advantage of using FA1/dlk for selection of cells is that dlk is a cell surface protein and selection can be performed by simple methods of antibody labelling, which result in the recovery of live cells which can be used for further purposes such as culturing.

The invention also concerns a method for preparing a cell population useful for transplantation that is enriched in FA1-expressing muscle mononuclear cells, satellite cells or muscle stem- or progenitor cells; which population may also be substantially free of other types of muscle cells.

The invention also in one aspect concerns the differentiation and/or transdifferentiation of such isolated myogenic stem/precursor cell into phenotypes distinct from the myogenic phenotype and used for replacement treatment. Myogenic, osteogenic and adipogenic differentiation is described in Asakura et al Differentiation 2001;68:245-253, which is hereby incorporated by reference. Another example would be (trans)differentiation along the hematopoietic lineages for replacement therapy in leukemia where isolated hematopoietic stem cells from the patient's own bone marrow may be complicated by contamination with cancer cells. Use of stem cells residing in the patient's muscle tissue would diminish the risk of contamination with transformed blood cells. Methods for differentiation of muscle-derived stem cells into hematopoietic cells is described in Asakura et al J. Biol Chem; 2002:123-134.

Differentiation of muscle stem cells or myogenic precursor cells may involve the use of growth factors and also the use of low oxygen level, (below 12%, preferably from 1 to 5% oxygen) as described in U.S. Pat. No. 6,184,035, has been shown to enhance the differentiation into skeletal muscle cells.

The invention also concerns therapeutic materials and methods for transplanting cultures containing cells of the invention that can be used in the treatment of myodegenerative diseases such as Duchenne's Muscular Dystrophy, and in the regeneration of muscle tissue following trauma, myocardial infarction or ischemia.

The present invention also provides cell populations enriched in FA1 expressing muscle mononuclear cells, myogenic progenitor cells or muscle stem- or progenitor cells, which are important vehicles for ex-vivo gene therapy. The cells may be obtained with the methods of the present invention. The cells may be encapsulated or implanted as naked cells.

These cell populations may also be used in drug screening, for the generation of cell-type specific antibodies and in gene discovery.

In a further aspect the invention relates to a method for measuring the content of FA1 expressing cells in a sample comprising the steps of:
a) contacting a population of cells selected from the group consisting of: a mixed population of mammalian muscle cells, populations of muscle stem- and/or progenitor cells, in vitro differentiated muscle stem and/or progenitor cultures; with labelled antibodies which bind specifically to FA1/dlk1,
b) optionally removing unbound antibodies and;
c) selecting cells labelled with the FA1 antibody,
d) quantifying the amount of selected cells resulting from step (c) relative to the quantity of cells used in step (a).

This method may be used to quantify the regenerative capacity of a cell population.

### BRIEF DESCRIPTION OF THE FIGURES

FIG. 1

Immunohistochemical staining for FA1/dlk1 in normal human fetal skeletal muscle at gestational week a) 15 and b) 21 c) Staining of neonatal (0 months) skeletal muscle. Arrows indicate mononuclear myogenic progenitor cells. Original magnifications, 400×

FIG. 2

Immunohistochemical staining for human FA1/dlk1 in an inflammatory myopathy shown at two magnifications. Original magnifications: a) 200× and b)

FIG. 3

FA 1/dlk1 localization in cryosections of rat skeletal muscle during regeneration of knife-cut lesioned muscle a)1, b) 3, c) 5, d) 7, e) 14 and f) 32 days after the lesion was inflicted. Original magnifications: 100×

### DETAILED DESCRIPTION OF THE INVENTION

The present invention concerns the use of antibodies that can bind to the FA1 antigen. More specifically it concerns the use of an antibody, referred to herein as “FA1 Ab” that facilitates the identification or isolation of specific populations of cells derived from mammalian muscle. These isolated cell populations make possible improved techniques for transplantation, drug screening and gene discovery. The isolated cells may also be employed to produce panels of monoclonal antibodies to specific populations of muscle-derived cells. Cells expressing FA1 are denoted FA1+ (FA1 positive) cells.

The isolated cell populations of the invention can also be employed in ex vivo gene therapy. The isolated cell populations may be further sorted based on the expression of other lineage specific markers. Examples include, but are not limited to; NCAM, VCAM1, Bcl-2, Pax7, Myosin, and M-cadherin. Co-selecting for one or more of these markers, improves the selection for myogenic precursor cells. Preferably the co-selecting also comprises a marker which is located on the cell surface, such as NCAM, VCAM 1, and Myosin.

The ability to recognize myogenic progenitor cells with antibodies allows not only for the identification and quantification of these cells in tissue samples, but also for their separation and enrichment in suspension. This can be achieved by a number of cell-sorting techniques by which cells are physically separated by reference to a property associated with the cell-antibody complex, or a label attached to the antibody. This label may be a magnetic particle or a fluorescent molecule. The antibodies may be cross-linked such that they form aggregates of multiple cells, which are separable by their density. Alternatively the antibodies may be attached to a stationary matrix, to which the desired cells adhere.

It is evident that selection of FA1 expressing cells can also be obtained using labelled probes that bind selectively to the mRNA sequence of FA1. As however this mRNA is present within the cell such labelling is more complicated than selecting for the antigen located on the surface of the cells and often would not be compatible with selecting viable cells because current methods for in situ hybridisation do not allow for the recovery in large numbers of living cells. A further, less preferred, embodiment would be to transform the muscle cells (preferably cultured cells) with a marker gene under the control of the FA1 promoter. Such a marker protein would be co-expressed with FA1 and could be used for selection. Examples of suitable marker proteins include but are not limited to surface proteins, CD8, influenza virus hemagglutinin, beta-galactosidase, green fluorescent protein, catachol 2,3-dioxygenase, and aeqourin. It suffices to use an expression construct which gives rise to transient expression; stable integration of the gene into the genome of the host cell is not required. Knowing the sequence of the coding region of FA1 it is possible for the skilled person to isolate the promoter sequence of FA1 and integrate it into such a construct.

In one embodiment, the present invention provides a method of selecting a population of cells derived from mammalian muscle; the method comprising: (a) providing a cell suspension from tissue derived from muscle (b) contacting said cell suspension with an antibody that binds the FA1 antigen; and (c) separating and recovering from said cell suspension the cells bound by said antibody, or (d) separating and recovering from said cell suspension the cells not bound by said antibody.

In a further embodiment the present invention provides a method of selecting a population of FA1 expressing cells derived from cultures of immature muscle cells; the method comprising: (a) providing a cell suspension prepared from a culture of immature muscle cells (b) contacting said cell suspension with an antibody that binds the FA1 antigen; and (c) separating and recovering from said cell suspension the cells bound by said antibody, or (d) separating and recovering from said cell suspension the cells not bound by said antibody.

In another embodiment the present invention provides a method of selecting a population of FA1 expressing cells derived from cultures of in-vitro differentiated immature muscle cells; the method comprising: (a) providing a cell suspension prepared from a culture of in-vitro differentiated immature muscle cells (b) contacting said cell suspension with an antibody that binds the FA1 antigen; and (c) separating and recovering from said cell suspension the cells bound by said antibody, or (d) separating and recovering from said cell suspension the cells not bound by said antibody.

Yet another embodiment of the present invention provides populations of mammalian muscle-derived cells enriched for FA1 expressing cells which are mono-nuclear cells, satellite cells, muscle progenitor cells, muscle stem cells, or immature muscle cells, which cultures may also be substantially free of other types of mammalian cells, as well as therapeutic methods employing such a cell suspension. The various FA1+ cell types may be present substantially alone or together with other FA1+ cells. Such cultures may be transplanted into a donor by surgical implantation, injection, or by intra-venous infusion.

In a further embodiment, the invention provides cell populations useful in methods of ex vivo gene therapy. Expression vectors may be introduced into and expressed in these cells, or their genome may be modified by homologous or non-homologous recombination by methods known in the art. In this way, diseases may be treated, which are related to the lack of secreted proteins including, but not limited to hormones, enzymes, and growth factors. Specific examples may include laminin, dystrophin and other factors known to affect muscle function. In one embodiment the cells may be used for treating mutations, i.e. introducing a functional gene to replace the mutated in cells isolated using the methods of the present invention. Inducible expression of a gene of interest under the control of an appropriate regulatory initiation region will allow production (and secretion) of the protein in a fashion similar to that in the cell that normally produces the protein in nature. A further embodiment provides populations of mononuclear myogenic cells that have been immortalized by insertion of an immortalizing gene such as a telomerase or vmyc.

According to another embodiment of the invention, FA1+ cells can be used in the production of monoclonal antibodies that recognize different antigens on mono-nuclear cells, myogenic progenitor cells or immature muscle cells. The cells isolated from muscle with FA1 antibody can be used as an immunogen, as described below, to produce a panel of monoclonal antibodies against mononuclear cells, myogenic progenitor cells or immature muscle cells or against sub-populations of such cells. These monoclonal antibodies may in turn be used to identify further mononuclear cells, myogenic progenitor cells or immature muscle cells and to divide FA1+ cells into subpopulations.

Antibodies that label the populations of mono-nuclear cells, myogenic progenitor cells or immature muscle cells and their differentiated progeny are extremely useful in drug screening, gene discovery and for transplantation purposes because they allow the enrichment of populations of such cells in a single step. Cells recovered with FA1 antibody derived from different stages in their development could be used in studies on the mechanisms of action of cells, factors, and genes that regulate cell proliferation and differentiation. Furthermore, myogenic progenitor cells from normal and pathological tissue may be recovered using FA1 antibodies and compared.

Production of Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide resembling the immunogenic protein, or a recombinantly expressed immunogenic protein. Furthermore, the protein may be conjugated to a second protein that is known to be immunogenic in the mammal being immunized. Examples of such immunogenic proteins include but are not limited to keyhole limpet hemocyanin, serum albumin, bovine thyroglobulin, and soybean trypsin inhibitor.

The polyclonal antibodies directed against the immunogenic protein can be isolated from the mammal (e.g. from the blood) and further purified by well known techniques, such as affinity chromatography, using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography.

Monoclonal anti-FA1 cell antibodies can be produced readily by one skilled in the art. The general methodology for making monoclonal antibodies using hybridoma technology is now well known in the art. See, e.g., M. Schreier et al., Hybridoma Techniques (Cold Spring Harbor Laboratory 1980); Hammerling et al., Monoclonal Antibodies and T-Cell Hybridomas (Elsevier Biomedical Press 1981); Kennett et al., Monoclonal Antibodies (Plenum Press 1980). Immortal, antibody-secreting cell lines can also be produced by techniques other than fusion, such as direct transformation of B-lymphocytes with oncogenic DNA or EBV. Several antigen sources can be used, if desired, to challenge the normal B-lymphocyte population that is later converted to an immortal cell line.

The FA1 protein is expressed as a cell-surface antigen on many immature cell populations. FA1 may also be purified from amniotic fluid as a 32-38 kD glycoprotein. A purification method for mouse FA1 is given by Bachmann et al., (J Reprod and Fert 1996; 107:279-285) and this can also be used for human FA1.

For example, the purified FA1 from amniotic fluid may be used as an immunogen to challenge the mammal (e.g., mouse, rat, hamster, etc.) used as a source for normal B-lymphocytes. The antigen-stimulated B-lymphocytes are then harvested and fused to an immortal cell line or transformed into an immortal cell line by any appropriate technique. A preferred hybridoma producing the monoclonal FA1 antibody is produced by challenging a mouse with the FA1 antigen and fusing the recovered B-lymphocytes with an immortal myeloma cell such as X63Ag8.6.5.3 or SP2/0-Ag14. Antibody-producing immortal cells can be screened for appropriate antibody production by selecting clones that are strongly and specifically reactive with the muscle myogenic progenitor cells using sectioned muscle tissue and immunohistochemistry. Antibodies produced by clones, which show those properties can then be tested for reactivity towards other cell populations known to express FA1.

A mouse hybridoma producing monoclonal FA1 antibody (clone 142.2) is described in a previous publication (Jensen et al., Eur J. Biochem. 1994 Oct. 1; 225(1): 83-92.). Other Hybridomas producing FA1 antibodies are: F12, F15, F30, F59, F31, F32, F33, F38, F54, 142-1. These hybridoma are all available from the University of South Denmark, Institute of Medical Biology, Winsløwsparken 21,1, DK-5000 Odense C, Denmark. These have been obtained using the methods described in Jensen et al (op.cit.). The immunogen to be used for the generation of antibodies against FA1/dlk1 may be 1) intact, native FA1 as purified from any human physiological fluid (milk, amniotic fluid, serum, seminal plasma, follicular fluid, urine); 2) FA1 or smaller products purified from primary cell cultures or cell lines (including genetically engineered cells) that generate soluble dlk1 forms; 3) membrane fractions from cells that express all forms of dlk1; 4) synthetic peptides or fusion proteins encompassing parts of or the entire extracellular part of the multiple dlk1 forms or; 5) chimeric proteins presenting any dlk1 form as a dimer, which includes fusion proteins and hybridoma cell lines in which the secreted immunoglobulin molecule has been genetically modified so the Fab region has been replaced with dlk1 in any form.

The antibodies according to the subject invention may be either monoclonal, polyclonal, or a mixture of monoclonal and/or polyclonal antibodies. The antibody may comprise whole antibody or antigen-binding fragments thereof, such as Fab₂, Fab and Fv fragments. Antigen binding fragments can be prepared using conventional techniques known in the art, such as proteolytic digestion of antibody by papain or pepsin, or through standard genetic engineering techniques known in the art. Monoclonal antibodies exemplified herein can be engineered so as to change the isotype of the antibody. For example, an IgG_{2A} isotype can be engineered as an IgG₁, IgG_{2B}, or other isotypes. Also contemplated by the subject invention are antibodies that are reactive with the FA1 antibody and which have been engineered to comprise human antibody constant regions. “Humanised” antibodies can be prepared using standard methods known in the art. See, for example, U.S. Pat. No. 5,585,089 (issued Dec. 17, 1996), the disclosure of which is hereby incorporated by reference. Also contemplated are single chain antibodies and chimeric antibodies.

Labelling of Antibodies

The antibodies of the subject invention can be labelled according to standard methods known in the art. Preferably, the label is one capable of providing a fluorescent signal. Fluorescence is preferred due to the high signal/noise ratio. For example, antibodies can be labelled with detectable labels such as fluorescein, rhodamine or with radioactive isotopes, or with biotin. Biotin binds strongly and irreversible to avidin. Biotinylated antibodies may be visualized by incubation with conjugates consisting of horseradish perioxidase and biotin bound to avidin followed by detection of the enzymatic activity using a chromogenic substrate. Alternatively, biotinylated antibodies may be incubated with a streptavidin-flurochrome.

Isolation of FA1 Expressing Cells

As indicated above, one application for antibodies to FA1 is the isolation of an enriched source of myogenic progenitor cells for transplantation into patients with Duchene's Muscular Dystrophy, or following trauma or myocardial infarction

The present invention contemplates the use of methods employing a FA1 antibody to separate muscle myogenic progenitor or progenitor cells from other muscle cells. The cells used for isolation include skeletal (striated) muscle, cardiac muscle and smooth muscle. These may originate from fetal tissue or adult tissue. The cells may also originate from cell cultures of raised from either of the above-identified cell types, such as cell cultures raised from embryonal stem cells.

Generally, a cell suspension prepared from mammalian muscle tissue by mechanical or enzymatic trituration is brought into contact with a FA1 antibody. Cells that have been bound by FA1 antibody are then separated from unbound cells by any means known to those skilled in the art. The muscle tissue may be taken from any muscular region or organ and may be selected by dissection. For instance it may be taken from skeletal (striated) muscle, cardiac muscle, or smooth muscle.

Various methods of separating antibody-bound cells from unbound cells are known. For example, the antibody bound to the cell (or an anti-isotype antibody) can be labeled and then the cells separated by a mechanical cell sorter that detects the presence of the label. Fluorescence-activated cell sorters are well known in the art. In one embodiment, the anti-FA1 antibody is attached to a solid support. Various solid supports are known to those of skill in the art, including, but not limited to, agarose beads, polystyrene beads, hollow fiber membranes, polymers, and plastic petri dishes. Cells that are bound by the antibody can be removed from the cell suspension by simply physically separating the solid support from the cell suspension. Preferred protocols, however, will be described.

Most of the isolation methods described and used in the art comprise a step of removing unbound antibodies prior to selecting cells. However depending on the sensitivity of the detection method, the strength of the signal originating from the label, and the number of FA1 molecules on the surface of the cells this step may not be possible. Important is that it is possible to distinguish FA1 expressing cells from background and from non-FA1 expressing cells.

Super paramagnetic nanoparticles may also be used for cell separations. The microparticles are coated with a monoclonal antibody for a cell-surface antigen. The antibody-tagged, super paramagnetic microparticles are then incubated with a solution containing the cells of interest. The microparticles bind to the surfaces of the desired cells, and these cells can then be collected in a magnetic field.

Selective cytophoresis can be used to produce a cell suspension from mammalian muscle containing myogenic progenitor cells. The cell suspension is allowed to physically contact, for example, a solid phase-linked monoclonal antibody that recognizes an antigen on the desired cells. The solid-phase linking can comprise, for instance, adsorbing the antibodies to a plastic, nitrocellulose, or other surface. The antibodies can also be adsorbed on to the walls of the large pores (sufficiently large to permit flow-through of cells) of a hollow fiber membrane. Alternatively, the antibodies can be covalently linked to a surface or bead, such as Pharmacia Sepharose 6 MB macrobeads. The exact conditions and duration of incubation for the solid phase-linked antibodies with the muscle cell suspension will depend upon several factors specific to the system employed. The selection of appropriate conditions, however, is well within the skill of the art.

The unbound cells are then eluted or washed away with physiologic buffer after allowing sufficient time for the stem cells to be bound. The unbound cells can be recovered and used for other purposes or discarded after appropriate testing has been done to ensure that the desired separation had been achieved. The bound cells are then separated from the solid phase by any appropriate method, depending mainly upon the nature of the solid phase and the antibody. For example, bound cells can be eluted from a plastic petri dish by vigorous agitation. Alternatively, bound cells can be eluted by enzymatically “nicking” or digesting an enzyme-sensitive “spacer” sequence between the solid phase and the antibody. Spacers bound to agarose beads are commercially available from, for example, Pharmacia.

The eluted, enriched fraction of cells may then be washed with a buffer by centrifugation and either said enriched fraction or the unbound fraction may be cryopreserved in a viable state for later use according to conventional technology or introduced into the transplant recipient.

The term ‘enriched’ is used to describe a population of cells in which the proportion of one particular cell type or the proportion of a number of particular cell types is increased when compared with the untreated population. According to one embodiment of the invention a population enriched in FA1+ cells comprises at least 5% FA1+ cells, more preferably at least 10%, more preferably at least 20%, more preferably at least 25%, more preferably at least 30%, such as at least 40%, for example at least 50%, such as at least 60%, for example at least 75%, such as at least 80%, more preferably at least 90%, more preferably at least 95%, more preferably at least 98%, such as substantially 100% FA 1+ cells.

The above cell populations containing FA1 enriched cells can be used in therapeutic methods such as cell transplantation, as well as other methods that are readily apparent to those skilled in the art. Other uses envisaged for these cells are for drug screening, antibody production and gene discovery. The compositions of the invention may also be used to generate antibodies to the membrane bound portion of dlk1, which remains following proteolytic cleavage of dlk1 to give the soluble form. They may also be used in methods to identify the protease responsible for cleavage of dlk1. For example dlk1 could be expressed in a eukaryotic cell (e.g. yeast) that does not normally process it. The eukaryotic cell could then be contacted with fractionated cell extracts from FA1 producing cells, and the fraction which cleaves dlk1could be identified and treated to isolate the said protease. The protease, which cleaves dlk1, could be a key element in the differentiation of primitive cell types. It is also envisaged that fractionated extracts containing the protease, obtained from enriched populations of FA1 producing cells of the invention, could be used to regulate the differentiation of stem- and progenitor cells.

It is understood that the initial medium for isolating stem cells/progenitor cells, the medium for proliferation of these cells, and the medium for differentiation of these cells can be the same or different. All can be used in conjunction with low or physiologic oxygen level culturing. The medium can be supplemented with a variety of growth factors, cytokines, serum, etc. Examples of suitable growth factors are basic fibroblast growth factor (bFGF), vascular endothelial growth factor (VEGF), epidermal growth factor (EGF), transforming growth factors (TGFα and TGFβ), platelet derived growth factors (PDGF's), hepatocyte growth factor (HGF), insulin-like growth factor (IGF), insulin, erythropoietin (EPO), and colony stimulating factor (CSF). Examples of suitable hormone medium additives are estrogen, progesterone or glucocorticoids such as dexamethasone. Examples of cytokine medium additives are interferons, interleukins, or tumor necrosis factor-α (TNFα).

In general, populations of FA1 positive myogenic progenitor cells may be used to establish primary cell cultures which can be expanded and used for transplantation, drug screening or any of the other purposes mentioned above. In one embodiment, populations of FA1 positive myogenic progenitor cells may be treated with the soluble FA1 antigen in order to maintain them in an undifferentiated state. This can be done with native human FA1 used in quantities from 1 to 10 μg/mL, such as approximately 5 μg/mL. Further details on the use of soluble FA1 to maintain an undifferentiated state are available in Hansen et al, Mol. Endocrinol 1998; 12:1140-49.

In another embodiment, the FA1 antibody can be used to isolate FA1 enriched cells, which can be used in various protocols of genetic therapy.

### EXAMPLES

The following examples are provided to illustrate specific embodiments of the present invention. The examples are included for illustrative purposes only, and are not intended to limit the scope of the present invention.

### Example 1

FA1/dlk1 in Human Fetal Muscle.

Human tissues: Normal fetal (n=6, gestational week 12-23) and neonatal (n=2, age=0 and 2 months) striated muscle tissue samples were obtained from the files at the Department of Pathology, Odense University Hospital. Formalin fixed and paraffin embedded human muscle specimens were cut in 5 μm sections, mounted on glass slides; air-dried and subsequently deparaffinized and re-hydrated. Endogenous peroxidase activity was blocked with H₂O₂ /methanol. Antigen-retrieval was performed by incubation with 0.05% (w/v) protease (Sigma, type XIV) in TBS at 37° for 15 minutes. Sections were incubated with a primary antibody (monospecific rabbit anti-human FA1) or a control antibody (primary antibody liquid-phase absorbed with affinity purified human FA1 as described in Jensen et al., 1993) diluted 1:100 (anti-human FA1) and subsequently reacted with a biotinylated secondary antibody (goat anti-rabbit IgG (DAKO E432, diluted 1:200)). The sections were then incubated with HRP-conjugated streptavidin (DAKO P397) diluted 1:300 and developed using 3-amino-9-ethylcarbazol as chromogen. Counter-staining was performed with haematoxylin.

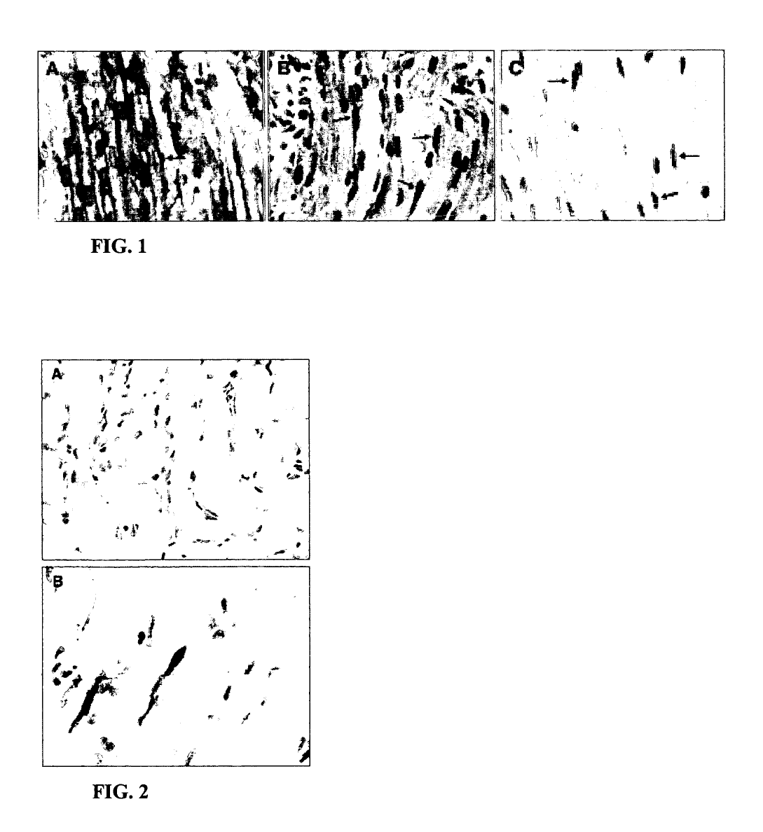

The sections demonstrated FA1 immuno-reactivity in the muscle fibers (FIGS. 1a and 1b). The intensity of the reaction decreased with increasing age. An accentuated perinuclear reaction was common. In addition, the mononuclear cells of spindle type situated both adjacent to the muscle fibers and more solitarily situated between the fibers expressed FA1. Their staining intensity was higher than that of the fibers and remained unaltered form 12 to 23 weeks of gestation, though a reduction in number seemed to take place.

In the neonatal muscle the muscle fibers were unstained but scattered, densely stained, mononuclear cells adjacent to the fibers were found; in contrast to earlier gestational ages where all such cells were FA1 immuno-reactive. Their position adjacent to the fibers suggests that they belong to the myogenic progenitor cell population (FIG. 1c).

### Example 2

FA1/dlk1 in Adult Human Striated Muscle.

Muscle tissue samples from 6 individuals with an inflammatory myopathy were obtained from the files at the Department of Pathology, Odense University Hospital. Normal adult skeletal muscle was obtained from biopsies with approval from the regional science ethical committee for Vejle and Funen counties.

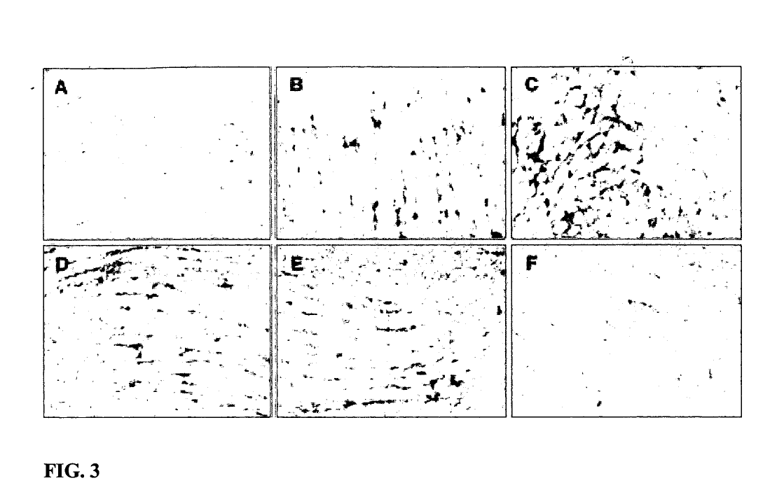

Tissues were formalin-fixed and paraffin embedded and stained as in example 1. In normal adult human skeletal muscle no FA1 immuno-reaction was observed. By contrast, in a series of 6 inflammatory myopathies all characterized by containing inflammatory infiltrates and necrotic and regenerating muscle fibers, mononuclear FA1 positive cells were present. They were located in close relation to apparently intact muscle fibers but not found at sites of necrosis (FIG. 2).

### Example 3

FA1/dlk1 Expression in a Rat Muscle Lesion Model.

Normal adult rat skeletal muscle was obtained from carbon monoxide (CO) intoxicated and decapitated Sprague-Dawley rats (M&B, Denmark). Animal experiment: Adult male rats (n=23) were deeply anaesthetized with pentobarbital and a knife cut lesion inflicted in the (thigh muscle). Animals were sacrificed by CO2-intoxication either 2 hours, 1, 3, 5, 7, 14, 32 or 56 days after the injury was inflicted and the lesioned muscle was removed. All rat specimens were quick-frozen in isopentane and stored at −70° C. until further analyzed. Cryosections (5 μm) of rat muscle specimens were air-dried overnight and fixed in acetone for 10 min at room temperature. Sections were incubated with a primary antibody (monospecific rabbit anti-rat FA1) or a control antibody (primary antibody liquid-phase absorbed with affinity purified rat FA1 as described in Jensen et al., 1993) diluted 1:2000 and subsequently reacted with a biotinylated secondary antibody (goat anti-rabbit IgG (DAKO E432, diluted 1:200)). The sections were then incubated with HRP-conjugated streptavidin (DAKO P397) diluted 1:300 and developed using 3-amino-9-ethylcarbazol as chromogen. Counter-staining was performed with haematoxylin.

Normal adult rat muscle contained single scattered FA1-positive cells in apposition to muscle fibers in some areas, but large areas were FA1-negative (not shown). However, we observed that knife-cut lesions induced an upregulation in the expression of FA1/dlk1 In order to investigate the time sequence of the FA1-induction in regenerating muscle a series of rat muscles with lesions varying from 2 h to 56 days were studied; Two hours after introduction of a cut lesion no FA1 immuno-reactivity had developed in relation to the lesion. However, at day 1 FA1-positive mononuclear cells situated along the damaged fibers appeared (FIG. 3a) and at day 3 an intense staining in these cells had developed in a zone around the lesion. A few cells of this type were found at the edge of the lesion but not within its center (FIG. 3b). At day 5 (FIG. 3c) the zone around the lesion containing FA1 immuno-reactive cells was narrower but more densely packed. Scattered less densely stained cells could be found at the rim of the lesion, but at the cut ends where fusion could be identified as newly formed muscle fibers with irregular contours and disorderly arranged nuclei, no FA1-positive mononuclear cells were found. The newly formed segments of muscle fibers were FA1-negative at this and any other age of lesion. Except for a further reduction of the peri-lesional zone containing FA1 immuno-reactive cells no changes were seen between day 5 and 7 (FIG. 3d).

At day 14 a reduction of the number of stained cells in the peri-lesional zone and also a reduction in their staining intensity could be observed. No FA1-positive cells were found in the fusion zone at this stage of regeneration (FIG. 3e).

At day 32 only few FA1 immuno-reactive cells remained detectable (FIG. 3f) and at day 56 all had disappeared (not shown). No structures except the mononuclear cells in apposition to the muscle fibers expressed FA1.

### Example 4

FA1/dlk1 Expression in Cultures.

Satellite cell cultures were established according to Gaster et al. (APMIS 2001:109(11):726-734). In brief, human muscle tissue was minced, washed, and enzymatically dissociated for 60 min. with 0.05% Trypsin-EDTA. Harvested cells were preplated in DMEM with 10% FCS and antibiotics on uncoated tissue culture plates for 30 min. Non-adherent cell were next transferred to culture dishes coated with 1% ECM-gel. After 24 hours, the medium was changed to DMEM supplemented with 2% Ultroser-G and 2% FCS. Before confluence was reached, the adherent cells were trypsinized, preplated as described for 30 min. and the non-adherent cells transferred to new coated culture plates. This procedure was repeated until the primary culture underwent four passages to ensure that all fibroblasts were removed form the cell population.

For studies on differentiating satellite cells, the established primary culture, devoid of fibroblasts, was plated onto non-coated tissue culture plates. At 75% confluence, the medium was replaced with basal medium (DMEM, antibiotics, and 25 pM insulin) containing 2% FCS (Gaster et al., APMIS 2001;109(11):735-744).

Primary and differentiated cultures were stained for FA1/dlk1 using the antibodies described. In addition, initially harvested cells and primary and differentiated cultures were analyzed by FACS using the same antibodies.

1. A method for obtaining a cell population enriched in cells selected from the group consisting of muscle mononuclear cells, satellite cells, muscle stem cells, and muscle progenitor cells, said method comprising the steps of
a) contacting a population of cells selected from the group consisting of:
a mixed population of mammalian muscle cells, and cultures containing or derived from muscle stem- and/or progenitor cells, with labeled antibodies which bind specifically to FA1/dlk1,
b) selecting cells labelled with the FA1 antibody.

2. The method of claim 1, further comprising the step of removing unbound antibodies prior to the step of selecting cells.

3. The method of claim 1 wherein the cell population is enriched in FA1+ muscle mono-nuclear cells.

4. The method of claim 1 wherein the cell population is enriched in FA1+ muscle satellite cells.

5. The method of claim 1 wherein the cell population is enriched in FA1+ muscle progenitor cells.

6. The method of claim 1, wherein the cell population is enriched in FA1+ muscle stem cells.

7. The method of claim 1 wherein the cell population is enriched in one or more FA1+ cell types, the cell types being selected from: muscle mononuclear cells; muscle satellite cells; muscle progenitor cells, and muscle stem cells.

8. The method according to claim 1, wherein the cell population obtained has been further selected for expression or lack of expression of at least one further marker.

9. The method according to claim 8, wherein the marker is selected from the group consisting of NCAM, VCAM1, M-cadherin, Bcl-2, Pax7 and Myosin.

10. The method according to claim 8, wherein the marker is NCAM.

11. The method according to claim 8, wherein the marker is Pax7.

12. The method according to claim 1, wherein the cells are human cells.

13. The method according to claim 1, wherein the cells are fetal cells.

14. The method according to claim 1, wherein the cells are taken from skeletal (striated) muscle, cardiac muscle, or smooth muscle.

15. The method according to claim 1, wherein the antibodies are fluorescently labelled.

16. The method according to claim 1, wherein the antibodies are magnetically labelled.

17. The method according to claim 1, wherein the antibodies are cross-linked.

18. The method according to claim 1, wherein the antibodies are biotin labelled.

19. A method for differentiation of myogenic stem/precursor cells comprising isolating FA1+ cells according to the method of claim 1, and further differentiating these cells into phenotypes distinct from the myogenic phenotype.

20. A method for transdifferentiation of myogenic stem/precursor cells comprising isolating FA1+ cells according to the method of claim 1, and further transdifferentiating these cells into phenotypes distinct from the myogenic phenotype.

21. The method of claim 20, wherein the cells are transdifferentiated along the hematopoietic lineage.

22. A composition of cells derived from mammalian muscle, which is enriched for FA1⁺ mono-nuclear cells/myogenic progenitor cells or muscle stem- or progenitor cells.

23. A composition of cells obtained by a process comprising the steps of:
a) contacting a population of cells selected from the group consisting of a mixed population of mammalian muscle cells, and cultures containing or derived from muscle stem- and/or progenitor cells, with labeled antibodies which bind specifically to FA1/dlk1,
b) selecting cells labelled with the FA1 antibody.

24. A genetically modified cell generated from the composition of claim 22.

25. The genetically modified cell of claim 24, which has been modified to express at least one hormone, enzyme, and/or growth factor.

26-35. (canceled)

36. A method for measuring the content of FA1 expressing cells in a sample comprising the steps of:
a) contacting a population of cells selected from the group consisting of: a mixed population of mammalian muscle cells, populations of muscle stem- and/or progenitor cells, in vitro differentiated muscle stem and/or progenitor cultures; with labelled antibodies which bind specifically to FA1/dlk1;
b) optionally removing unbound antibodies;
c) selecting cells labelled with the FA1 antibody; and
d) quantifying the amount of selected cells resulting from step (c) relative to the quantity of cells used in step (a).

37. The method according to claim 36, further comprising the step of selecting the cells for expression or lack of expression of at least one further marker.

38. A method of identifying mono-nuclear myogenic cells comprising contacting the cells with an antibody to FA1/dlk1 and imaging the antibody.
